(19) United States
(12) Patent Application Publication
(10) Pub. No.: US 2023/0173190 A1
(43) Pub. Date: Jun. 8, 2023

(54) CAPSULE, DEVICE, SYSTEM AND METHOD FOR AUTOMATICALLY-CONTROLLED PARENTERAL ADMINISTRATION OF A MEDICAMENT PERFORMED BY INJECTION

(71) Applicants: Armando Miguel BARBOSA DE ABREU E SOUSA, Caldas da Rainha (PT); Miguel Afonso BARBOSA DE ABREU SOUSA E BATISTA, Vieira de Leiria (PT); Jens HOLM, Caldas da Rainha (PT)

(72) Inventors: Armando Miguel BARBOSA DE ABREU E SOUSA, Caldas da Rainha (PT); Miguel Afonso BARBOSA DE ABREU SOUSA E BATISTA, Vieira de Leiria (PT); Jens HOLM, Caldas da Rainha (PT)

(73) Assignee: Armando Miguel BARBOSA DE ABREU E SOUSA, Caldas da Rainha (PT)

(21) Appl. No.: 17/997,883

(22) PCT Filed: May 4, 2021
(86) PCT No.: PCT/IB2021/053700

§ 371 Date: Nov. 3, 2022

(30) Foreign Application Priority Data
May 6, 2020 (IT) 102020000010015

(51) Int. Cl. A61M 5/32; A61M 5/46; A61M 5/28; A61M 5/142

(52) U.S. Cl. CPC A61M 5/3234; A61M 5/46; A61M 5/282; A61M 5/14244; A61M 2205/273

(57) ABSTRACT

An injection capsule for medical use, having:
a closed main body having an internal reservoir receiving a medical substance in liquid form to be administered to a target subject; and
a needle, having one or more inlet bores located at its lateral skirt and configured to allow the inlet of the medical substance within its tubular body. The needle is activatable between a rest configuration, wherein it is housed within the reservoir and immersed in the medical substance, and an injection configuration, wherein it is pushed outside the closed main body to administer the medical substance by injection to the target subject.

### FIELD OF THE INVENTION

The present invention relates to devices, methods and systems for the parenteral administration of one or more medicaments, in particular through injection.

### BACKGROUND OF THE INVENTION

Several health authorities worldwide report that there is an increasing number of situations of (sudden) human death - or risk of severe physical and cognitive damages - that could be avoided by a timely parenteral administration of a medicament.

The majority of the situations mentioned above are associated with the following main groups of individuals:
patients with identified cardiovascular problems, and in high risk of severe cardiac problems or strokes, including sudden cardiac death;
people without any precedent known cardiovascular disease, strokes or other known potential cause of sudden death, and who incur in unexpected situations of sudden death without an easily identifiable root cause;
people with severe allergies suffering situations of fatal, or potentially fatal, anaphylactic shock resulting from the exposure to specific allergenic agents;
people suffering envenomation from natural fauna or flora, eventually in combination with allergies;
civil or military individuals exposed to chemical or biologic toxic substances;
people doing sports, or physical activity, reaching their (unknown) body’s maximum physical limits, leading to situation of sudden death.

In the vast majority of the above cases, several symptoms occur before reaching situations of high risk of death or of serious physical damages, in particular to the brain, heart and/or nervous system. However, often symptoms are not perceived or understood by the target subject. In other cases, the cause of injury or sudden death acts very rapidly, without leaving any reaction time, either mental or physical, for said target subject.

Therefore, in the situations discussed above the voluntary administration of a medicine - particularly enterally, e.g. by oral tablets - is not an available or resolutive option, either because it involves physical and mental energies that the subject has not or because the subject does not carry with him/her the necessary medicament for a timely administration.

### SUMMARY OF THE INVENTION

The technical problem underlying the present invention is therefore to provide a product, device, system and method to address the situations mentioned above with reference to the state of the art.

The above problem is solved by an injection capsule according to claim 1 and by a device according to claim 22.

Preferred features of the invention are the object of the dependent claims.

The invention provides technical solutions to enable the parenteral administration of medication, via injection, in any case of (urgent) need. Advantageously, according to preferred embodiments, the administration can occur automatically, i.e. in an unassisted way, in a timely and controlled manner and with a specific dose of a defined medication.

Alternatively, in different embodiments, the administration can occur with a simple action or gesture by the target subject, still assuring timely and proportionate intervention.

According to some embodiments, detecting means, e.g. sensors or transducers, of a specific physical or mental condition can determine and/or control the medicament administration and/or provide a signal to the target subject about the need for said administration.

The basic component of the device, assembly, or system according to the invention is a packaged medicament in form of an injection capsule or cartridge. The latter is, advantageously, configured as a consumable, i.e. it is a disposable item. The medicament is received in a casing, or container, including one or more compartments, or reservoirs, for receiving one or more medical substances, for example in liquid form or capable of being reduced in liquid form at the administration time.

The injection capsule may also include one or more needles that are selectively activatable, i.e. advanced or anyway moved towards a target subject skin, to inject the medicament in the body of said target subject. The needle(s) and/or its/their activation means can be housed in the same casing as the medicament.

Generally speaking, the packaged product can include one or more compartments, casings or containers, each housing one or more medical doses of the same or different medical substances and/or one or more needles.

Advantageously, the needle(s) and/or the medical substance(s) are sterile and/or stored in fully hermetic conditions. Generally speaking, the needle(s) and/or the medical substance(s) are isolated from potential external contamination and/or (chemical) adulteration/degradation. Preferably, the or each reservoir, casing, container or compartment receiving the medical substance(s) and/or the needle(s) is isolated from natural or artificial light, to avoid a potential damaging impact of infrared or ultraviolet radiation.

The needle(s) can be housed beyond one or more frangible or pierceable wall, apt to be crossed by the same needle(s) upon its/their activation.

Structural means that holds the needle(s) in place before and/or during such activation can also be provided.

In specific embodiments, one or more elastic or magnetic elements, such as a spring element, may be provided for the purpose of returning the needle(s) and, generally speaking, the capsule to the original configuration or position at the end of the administration of the medication by injection.

The packaged medicament may also incorporate a thin reservoir that contains a liquid, or gel, with disinfecting and/or anaesthetic properties. The latter may be embedded in a thin layer, e.g. of High Retention Material (HRM), which is trespassed by the needle(s) upon use.

In an embodiment, said substance with disinfecting and/or anaesthetic properties may be provided as impregnation of one or more layers of a pierceable or frangible wall. The latter may be shielded by a peeling foil or element that is removed upon application of the device to a target subject, so as to allow the substance to act in contact with the epidermis.

The packaged product, in specific embodiments, is received within, or is part of, an activation device or module, the latter preferably including a housing, e.g. a cavity, to hold and secure the packaged product. Preferably, the packaged product is housed reversibly in the module, so that it can be removed, and eventually disposed off, after use.

The activation device can be configured to determine a (relative) movement of the needle(s) to perform the injection.

The activation module is preferably received, or housed, in a wearable device, e.g. a smart watch, a jacket, a garment in general, a bracelet or the like. Preferably, the wearable device is used close or attached to the skin, in a location of the human body that enables to administrate an injection in an effective way.

The activation module or the wearable device, or even the packaged product, can incorporate a control unit. In particular, the activation module, the packaged product and/or the wearable device can be configured to administer the injection upon reception of a predetermined activation signal triggered by the control unit, which signal may be electric, electronic, electromagnetic, wireless in general (e.g. Bluetooth) and/or mechanic.

The control unit may be associated with the aforementioned sensors or transducers for detecting a medical condition of the target subject.

The activation module can alert the target subject that an injection is about to be administrated, according to the pre-programming of the control unit by the user and/or medical doctor. As a standard mode, the administration by injection takes place according to one or more pre-defined programmed conditions required to trigger such action.

The control unit may also comprise means for communicating/interfacing with other activation modules, wearable devices or control means.

According to preferred modes of operation of the system of the invention, one or more symptoms of urgent need of administration of a medication are detected in real-time via sensors, transducers and/or other means, e.g. incorporated in devices to collect bio-data / health-data, which can be wearable devices, such as a smart-watch used in the wrist, or devices attached to other parts of human body, such as the torso or legs. Such detection means may interface with other devices, such as a smart-phone or other portable devices incorporating features for the purpose of collecting, receiving and treating bio/health data. The devices can remotely interface with each other, as well as with a central control unit for the purpose of data analysis, decision making, and data storage.

The control unit, based upon the bio/health data collected, triggers actions in case of need, following automatic, pre-programmed decision-making protocols.

In specific embodiments, the invention provides a fully encapsulated consumable, device and system, for automatic unassisted parenteral medication performed by injection.

Advantageously, the device, product, system and method of the invention allow:
detecting in real-time symptoms and general health conditions of its users, monitoring key parameters of their health conditions, detecting potential situations of sudden death and/or of related, or similar, conditions;
acting autonomously, and automatically, in real-time according to defined protocols, to perform parenteral administration via injection in a fully automatic and unassisted way, always in full sterilized conditions, of a medication of any type;
remotely communicating to a defined list of people, databases and/or entities the specific detection/occurrence and the type of medication parenterally administrated; and/or
alerting the target subject about the occurrence and the situation risk, providing specific adequate instructions that the subject may take to self avoid, or minimize, a worsening of his/her health conditions.

In conjunction with this latter issue, the device and system may act as a paramedical companion during the time between the administration of the medication and the first aid, and/or further specialized medical assistance, namely monitoring key health parameters of the subject and providing instructions accordingly, namely:
to guide the subject to position the body in specific ways;
to guide the subject to perform specific patterns of breathing, in terms of inspiration, expiration, duration and frequency, according to key/vital health parameters monitored in real-time;
to guide the subject to follow specific mental stages of psychological comfort that may lead to psychological/mental and physical pain relief, including self-induced hypnotherapy and similar alternative approaches.

Therefore, according to embodiments of the present invention, technical solutions are provided that enable a timely automatic action - after the detection of symptoms via health data collection - by triggering immediate delivery of a parenteral administration of a medication, via sterilized injection, in an effective and controlled way.

The invention allows to radically change the standard way parenteral administration of medication via injection is performed in relevant art, usually using standard syringes, needles, related apparatus, disinfecting operations and associated medical procedures and personnel.

In fact, there is no need for a solid syringe with a plunger a sealing element, which can be replaced by a hermetic reservoir that contains the needle. Therefore, there is no need for the extra packaging usually existing for the syringes and the needles and of all the specific procedures to effectively apply an injection by conventional means, which usually requires a medical or paramedical professional.

On a sustainability perspective, namely in terms of environmental impact, the proposed concept of all-in-one consumable strongly avoids the use of traditional plastics, like for example all the different polymeric compounds used in syringes, plungers, sealing elements and needles sets, as well as in the packaging of all those parts.

The product, device and system of the invention can be very simple to operate, be reliable and have competitive manufacturing cost.

Other advantages, features and use modes of the present invention are explained, or will be evident, in the following detailed description of several embodiments, provided by way of example and not for limiting the scope of protection.

### BRIEF DESCRIPTION OF THE DRAWINGS

Reference will be made to the figures of the annexed drawings, wherein:

FIGS. 1 and 2 show each a schematic lateral view, partially in longitudinal section, of an injection device according to a first preferred embodiment of the invention, in a rest and an injection configuration, respectively;

FIGS. 3 and 4 show a schematic perspective view of the device of FIG. 1 in a rest condition and during manipulation by a user, respectively;

FIG. 5 shows a perspective view of a capsule, or cartridge, for use with the device of FIG. 1;

FIG. 5A shows a perspective view of a variant of the injection capsule, or cartridge, of FIG. 5;

FIG. 6 shows a longitudinal sectional and exploded view of the capsule of FIG. 5;

FIG. 6A shows a perspective view of a component of the device of FIG. 1;

FIGS. 7A to 7C relate to an embodiment of a needle retaining element of the capsule of FIGS. 5 and 5A, showing a plan, lateral and transverse sectional view thereof, respectively;

FIGS. 8A and 8B show each a schematic sectional view of a respective variant of a needle retaining element of the capsule, or cartridge, of FIGS. 5 and 5A;

FIGS. 9 and 10 show each a schematic lateral view, partially in longitudinal section, of an injection device according to a second preferred embodiment of the invention, in a rest and an injection configuration, respectively;

FIG. 11 shows a schematic perspective view of the device of FIG. 9 in a rest condition;

FIG. 12 shows a schematic lateral view, partially in longitudinal section, of a capsule, or cartridge, according to another preferred embodiment of the invention;

FIG. 13 shows a longitudinal sectional and exploded view of the capsule of FIG. 12;

FIGS. 14A to 14C show each a longitudinal sectional view of the capsule of FIG. 12, in a rest, injection and recovery configuration, respectively;

FIG. 15 shows a longitudinal sectional and exploded view of a capsule, or cartridge, according to a variant embodiment of the invention;

FIGS. 16A and 16B refer each to a thrust and perforation element of the capsule, or cartridge, of FIG. 15, showing a plan and transverse sectional view thereof, respectively;

FIGS. 17A to 17D show each a longitudinal sectional view of the capsule of FIG. 15, in a rest, a first injection stage, a second injection stage and a recovery configuration, respectively;

FIG. 18 shows a longitudinal sectional and exploded view of a capsule, or cartridge, according to a further preferred embodiment of the invention;

FIGS. 19A to 19C show each a longitudinal sectional view of the capsule, or cartridge, of FIG. 18, in a rest, a first injection stage and a second injection stage configuration, respectively;

FIGS. 20A and 20B refer to a device according to any of the embodiments of the above figures during an operation of insertion of a capsule, or cartridge, according to FIG. 5A, showing each a perspective view from below during introduction and holding of said capsule, respectively;

FIGS. 21A, 21B and 21C refer to a device according to any of the embodiments of the above figures during an operation of insertion of a capsule, or cartridge, according to FIG. 5, showing each a perspective view from below during capsule introduction, holding and peeling off of a film of said capsule, respectively;

FIGS. 22A and 22B show each a schematic lateral view of a respective variant of a curved needle that is included in embodiments of the present invention;

FIGS. 23 and 24 show each a schematic lateral view, partially in longitudinal section, of a respective variant of a capsule or cartridge that includes the needle of FIG. 22B;

FIG. 25 shows a schematic perspective view of the capsule, or cartridge, of FIG. 24, in a rest configuration;

FIG. 26 shows a schematic sectional view of the capsule, or cartridge, of FIG. 24, in an injection configuration;

FIGS. 27 and 28 show each a longitudinal sectional view of another embodiment of a device according to the present invention that includes the capsule, or cartridge, of FIG. 24, in a rest and injection configuration, respectively;

FIGS. 29, 30 and 31 refer to a further embodiment of the device according to the invention, showing a schematic perspective view, a longitudinal sectional view in a rest configuration and a longitudinal sectional view in an injection configuration thereof, respectively;

FIGS. 32 and 33 relate to a still further embodiment of the device of the invention, showing each a longitudinal sectional view in a rest and an injection configuration thereof, respectively;

FIG. 32A shows a perspective view of a component of the device of FIG. 32;

FIGS. 34 and 35 show a schematic perspective view of the device of FIG. 32 in a rest condition and during manipulation by a user, respectively;

FIG. 36 shows a perspective view, partially in transparency, of a capsule, or cartridge, according to another variant embodiment of the invention;

FIGS. 37A and 37B show each a longitudinal sectional view of the capsule, or cartridge, of FIG. 36, in a rest and an injection configuration, respectively;

FIGS. 38A and 38B show each a schematic longitudinal sectional view of an embodiment of the device of the invention including the capsule, or cartridge, of FIG. 36, in a rest and an injection configuration, respectively;

FIG. 39 shows a perspective view of a capsule, or cartridge, according to a further variant embodiment of the invention;

FIGS. 39A to 39D refer to the capsule, or cartridge, of FIG. 39, showing a perspective exploded view partially in transparency, a plan view, a longitudinal sectional view and a perspective view of a portion thereof, respectively;

FIGS. 40A and 40B refer to a wearable item including a device according to an embodiment of the present invention, showing a plan view from below and a sectional view thereof, respectively.

### DETAILED DESCRIPTION OF PREFERRED EMBODIMENTS OF THE INVENTION

Several embodiments and variants of the invention will be described below, with reference to the figures already introduced.

Generally speaking, analogous components are indicated in all the various figures using the same reference number.

Further embodiments and variants other than those already described will be explained solely in conjunction with the relevant differences.

Moreover, the features of the various embodiments and variants described below are to be understood as combinable, where compatible.

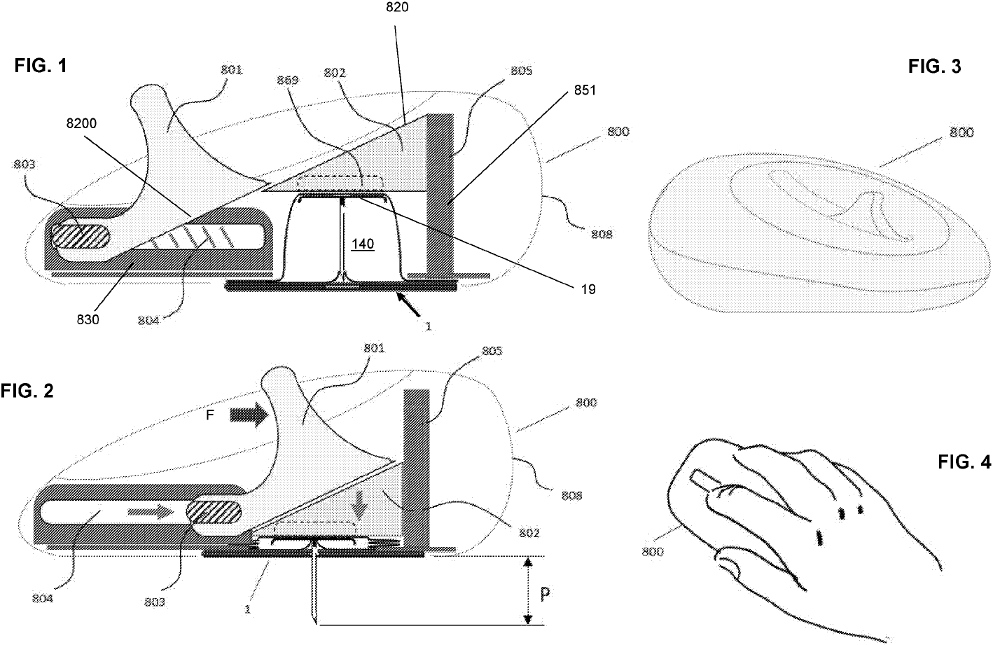

Referring initially to FIGS. 1 to 4, an injection device according to an embodiment of the present invention is globally denoted by 800. The injection device 800 has a device body 808, frame or housing, which receives at least one injection capsule 1 for medical use.

In the present embodiment, the device body 808 is configured substantially like a computer mouse, to enable precise and stable ergonomic use with a single hand, as better appreciated from FIG. 3.

The injection device 800 comprises activation means 801, in form of a slidable lever or handle, the functioning of which will be explained shortly below.

The slidable lever 801 can be activated by a human hand or other body district, as shown in FIG. 4, or be driven automatically by other technical means upon a triggering signal provided by a local or remote control unit. The slidable lever 801 has a guidance portion 803 slidably received within a lever guide member 830.

As shown in FIG. 1, the injection device 800 also includes push means 802 configured to act upon the injection capsule 1. The push means 802 are slidable according to a vertical direction and to this end slidably coupled to a push guide member 805.

Variant embodiments may provide a bilateral arrangement, or duplication, of guide members 830 and 805.

The injection device 800 also comprises recovery means to bring the capsule 1 and the device itself in an initial configuration after injection.

In the present embodiment, such means comprises elastic elements provided at the guide members 830 and 805 and therein denoted by 804 and 851, respectively.

Still in the present example, the recovery means comprises a device magnetic element 869, e.g. in form of a disc housed inside the push means 802 and shown also in FIG. 6A.

The magnetic disc 869 is coupled to a capsule magnetic element, the latter associated with a needle-holding, or retaining, member 19 and which may also consist in making the whole holding member or a part thereof of a magnetic material, or a material that is attracted to magnets and/or to an electromagnetic field, e.g. ferromagnetic.

The device magnetic element 869 may be a permanent magnet or an electromagnet, and the capsule member 19 may be made of a material that is attracted to magnets or to an electromagnetic field.

With reference also to FIG. 6, the injection capsule 1 comprises a closed main body 14 defining a container having an internal reservoir 140.

The internal reservoir 140 receives a medical substance in liquid form to be administered to a target subject.

The main body 14 is defined by a body member 141, or shell, substantially shaped as an inverted cup, preferably with a truncated cone or cylindrical shape. The body member 141 is hermetically closed at a bottom thereof by a pierceable base structure 146, preferably a film structure of a multilayer type. The pierceable base structure 146 is apt to be rested, in use, onto the target body skin and to be perforated, upon need, by a needle 20.

The main body 14, and particularly its body member 141, has a collapsible structure configured to assume a minimal encumbrance configuration, the latter shown in FIG. 2.

The body member 141 can be made of laminated materials, namely polymeric foil(s) and/or metallic foil(s), and/or foils of materials coated with polymeric or metallic materials.

The body member 141 can be produced by existing standard blistering technologies using laminated thin foils, as generally known, e.g., in the field of medication packaging, and other existing manufacturing technologies used to expand and form such materials.

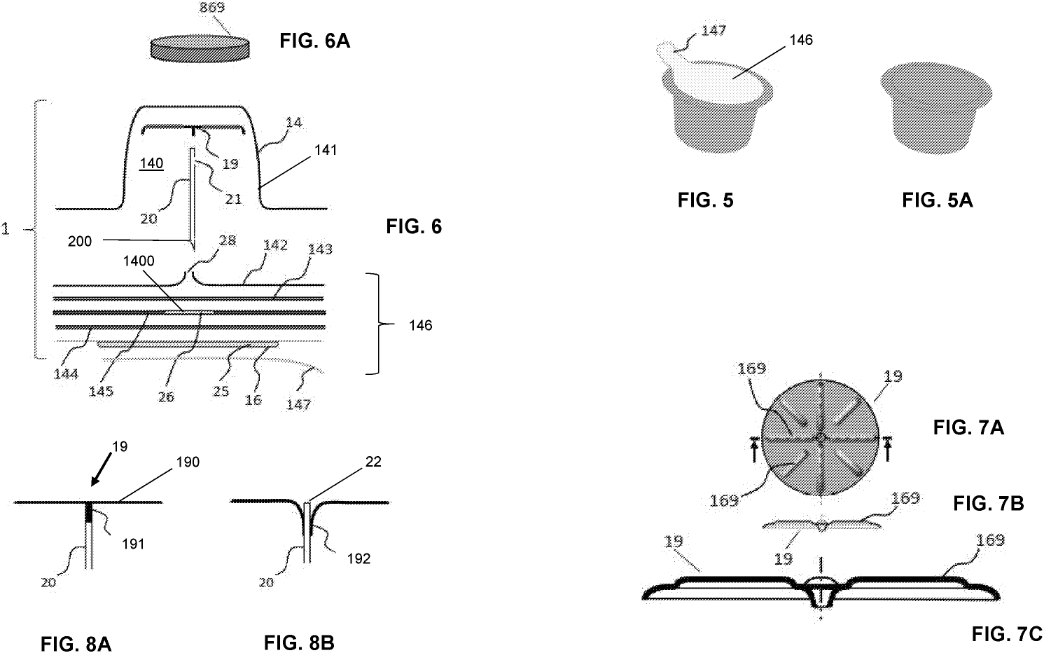

As shown in FIG. 5, the capsule main body is configured substantially as a coffee capsule or cartridge. Such body may have a frusto-conical shape. This design is particularly adequate for manufacturability, due the conical angle that enables the easy removal from cavities in manufacturing tools as well as easy fit and insertion in cavities of the devices that use capsules or cartridges shaped in such way.

The base structure 146 may include an external layer 147 peelable before use and advantageously provided with a lateral peeling tab. The external layer 147 may be made of a polymeric film or of a laminated structure including, e.g., a polymeric material and a metallic foil.

The external peelable layer 147 may be used to shield an inner layer, such as incorporating a HRM (High Retention Material), impregnated with a disinfectant and/or with an anaesthetic substance, which acts in contact with the skin when the injection capsule 1 is in use. The user of a device using a capsule with such feature may remove the peelable layer 147 when the capsule is already inserted and well positioned in its compartment in the device, and disinfect the region of skin where the injection will be provided, before triggering the activation of the capsule to perform the injection.

In FIG. 5A, a variant embodiment is shown wherein said peelable layer is not provided (or the same capsule of FIG. 5 after removal of the peelable layer 147).

A preferable construction of the film structure 146 as shown in FIG. 6 includes one or more of the following elements, eventually in addition to external layer 147 and preferably according to the sequence shown in said FIG. 6:
a core layer, or foil, 16, preferably made of polymeric compound(s) and which may be impregnated by a disinfectant and/or with an anaesthetic substance 25;
a first inner layer, or foil, 144;
a second inner layer, or foil, 145, preferably having an aperture, or opening, 26;
a third inner layer, or foil, 143;
a terminal inner layer 142, preferably having a perforation 28.

The above layers can be coupled one another by lamination.

The aperture, or opening, 26 of the second inner layer 145 may act as a guide for the needle 20, assuring its positioning in all conditions of the injection capsule 1. The opening 26 is enclosed, i.e. not accessible, when the layer 145 is laminated together with foils 143 and 144, creating a specific compartment which can be empty, with air or vacuum, or alternatively can be used as a small reservoir 1400 containing a disinfectant or anaesthetic. Such reservoir 1400 is trespassed by the needle 20 just before penetrating in the skin, therefore spreading a portion in the skin surrounding the needle itself.

In an exemplary embodiment, layer 145 is made of a polymeric and/or metallic material.

Alternatively, or in combination with reservoir 1400, for the purpose of disinfecting and/or anaesthesia the core layer 146 can act as a planar reservoir, as mentioned above in conjunction with peelable layer 147.

Containment layers 143 and 144 can be made of an elastomer polymeric material alone and/or laminated with a graphene-based compound.

Generally speaking, all the above layers, and particularly layers 142 to 145, may include an elastomeric material, which enable a hermetic effect when crossed by the needle 20 against the lateral skirt of the needle itself.

Inside the reservoir 140, and generally immersed in the medical substance, the aforementioned needle 20 is provided. The needle 20 has a tubular body defined by a lateral skirt, it too denoted by 20. The tubular body has a dispensing outlet bore 200 at a longitudinal terminal end thereof and at least one inlet bore 21 located at said lateral skirt and configured to allow the inlet of the medical substance within the tubular body.

The injection capsule 1 comprises the aforementioned needle-holding, or retaining, member 19, arranged at an opposite longitudinal end with respect to said dispensing bore 200 and preferably having a substantially plate-like or disc-like shape. The needle holding member 19 may have a porous structure.

In particular, as better shown in FIG. 8A, the needle holding member 19 has a retaining plate 190 from which a stem 191 protrudes, preferably at a central portion of the retaining plate 190. The stem 191 engages externally the needle tubular body and retains it in place.

The needle holding member 19 is attached at the internal surface of a top base of the body member 141.

The holding member 19 assures correct position of the needle 20 in any conditions inside the injection capsule 1 as well as when operating.

FIG. 8B shows a possible variant embodiment for the needle holding member 19, wherein the needle 20 is peripherally engaged at its external surface of such terminal end by a central tubular protrusion 192 that retains it.

In such alternative embodiment of FIG. 8B the needle 20 keeps its tubular shape open at its terminal end, which can therefore be also used as one inlet bore 22. This latter option operates, in particular, in the configuration of FIGS. 7A to 7C, wherein the needle holding member 19 incorporates radial ribs 169 enabling adequate fluid mechanics during operation, acting as medicament channels towards the axial inlet bore 22.

FIGS. 7A to 7C relate to an exemplary implementation of the needle holding member 19 as including the aforementioned radial ribs 169 extending onto retaining plate 190. The latter may be made of a material that is attracted to magnets, and/or attracted to electromagnetic generated fields, and may include magnetic material, to couple with device magnetic element 869. As said above, this exemplary design incorporating the radial ribs 169 assures adequate fluid mechanics during the use of the capsule, as well as keeping close predominantly planar interface of the holding member 19 with device magnetic element 869.

The needle 20 is activatable between a rest configuration, shown in FIG. 1, wherein it is housed within the reservoir 140 and immersed in the medical substance, and an injection configuration, shown in FIG. 2, wherein it is pushed outside the closed main body 14 to administer the medical substance by injection to the target subject.

The passage from the first to the second configuration is determined by the aforementioned activation means 801 of the injection device 800, which act upon the aforementioned push means 802. In particular, the guidance portion 803 moving inside guide member 830 according to an horizonal translational movement. An inclined surface 8200 of the slidable lever 801 engages in abutment a correspondingly inclined surface 820 of the push means 802. As a consequence of such coupling, the push means 802 moves downwards according to a vertical movement guided by the vertical guide member 805. Such downward movement determines the collapse of the capsule body member 141 and penetration of the needle 20 through the film structure 146 and the subject skin. The depth of penetration is exemplified in FIG. 2 and denoted by P. Once the body member 141 collapses, and the needle 20 perforates, or pierces, structure 146, the overall assembly of the injection capsule 1 works like a pump, powered by force F applied to lever 801.

Once the action upon lever 801 is terminated, the recovery means brings the capsule 1 and the device 800 in their original configuration, with the needle 20 retracted inside the collapsible body member 141.

Therefore, at the end of its operation the device 800 contains and retains the used consumable 1 inside its housing 808, with the needle 20 safely inside its compartment 140, and the consumable 1 can then be manually pulled from the housing 808 to be safely discarded.

At no moment, before, during or after operation, the needle is exposed to external contact or access.

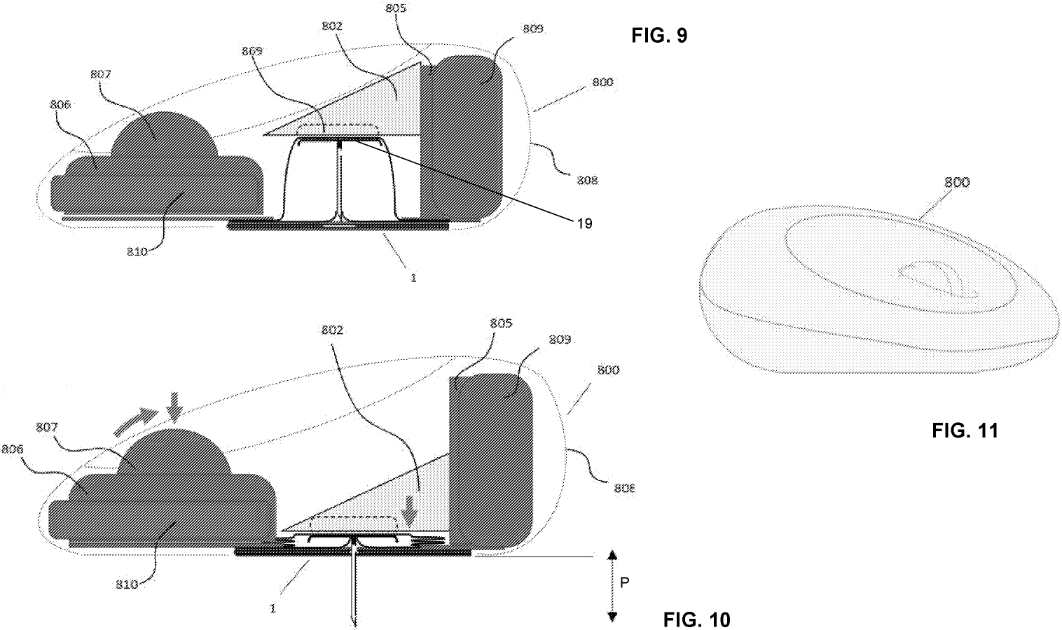

FIGS. 9 to 11 show another embodiment of the invention, which, as anticipated, will be described only in conjunction with the features differentiating it with respect to the previous embodiments or variant embodiments.

The injection device, denoted as 800 also in this case, includes activation means, denoted by 807, protruding from a device body 808. The activation means 807 may be in form of a button that can be pushed and/or rotated forwards or backwards. Such movement is detected by, or transmitted to, an electronic switch 806, or an equivalent means, which translates the movement of the activation means 807 into a command for another command member 809 associated with wireless communication means 810. The command member 809 may be, for example, an electric/electronic motor/driver that drives the upwards and downwards movement of the push means 802, the latter coupled to guide member 805.

Also in this case recovery means can be provided, in particular as associated with device magnetic element 869 and possibly needle-holding, or retaining, member 19.

The injection device 800 of the present embodiment, as electrically/electronically driven, may be more independent from the user in terms of action applied, in particular for the timing of the administration.

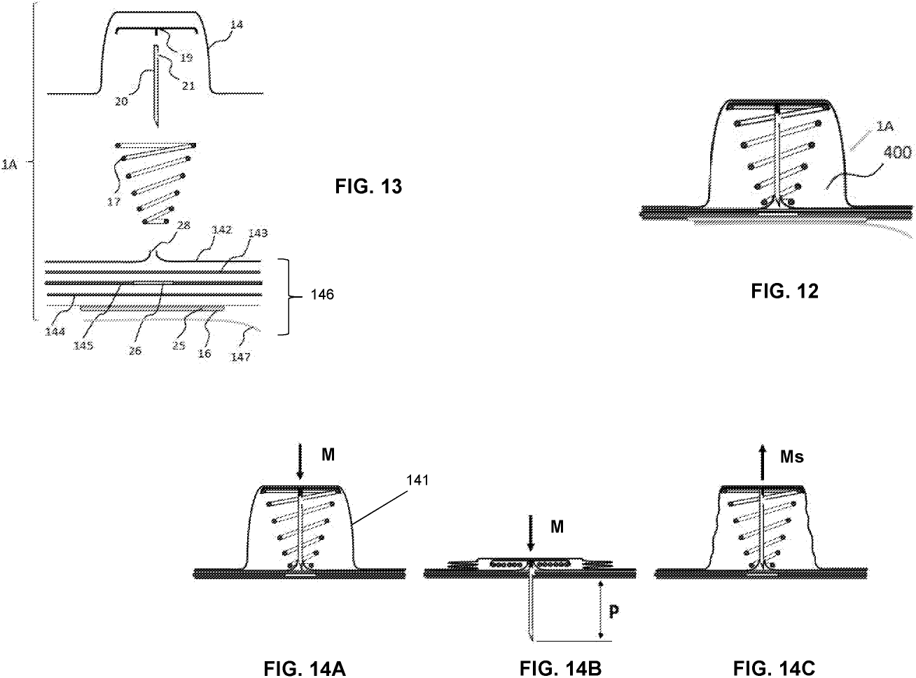

FIGS. 12 to 14C show an exemplary variant embodiment of an injection capsule, denoted as 1A, that can be used in any of the injection devices described above.

The capsule 1A differs from the previous embodiments in that it includes, inside an internal reservoir 400, a contrast means 17, for example in form of a spiral spring. The latter is arranged circumscribed to the needle 20, with its larger section winding in abutment at the inner side of retaining plate 190 of needle holding member 19. The contrast means 17 can also be fastened to member 19 in a mechanical way, including soldering. The contrast means 17 can be made of metallic or polymeric material(s), or comprise both types of materials.

The contrast means 17 acts as a further recovery means allowing collapsible body member 141 to recover its original shape after injection, as shown in the sequence of FIGS. 14A-14C. When a force M is applied, e.g., through the push means, the capsule shell 141 starts collapsing while pressure is applied to the spring element 17 and the needle holding member 19, moving the needle element 20 down and making it perforating the skin up to a defined total penetration P. During such process, the liquid contained is compartment/reservoir 400 of the capsule 1A is injected through the needle 20. When the applied force M stops, the spring element 17 is released, and it moves up recovering its original shape by means of recovery action Ms, pushing the holding plate 190 up, which also reshape the walls of the capsule body member 141 mostly to its original shape. This action also retracts the needle 20 up, extracting it from the skin/body, and placing it back to its original housing and safely keeping it there.

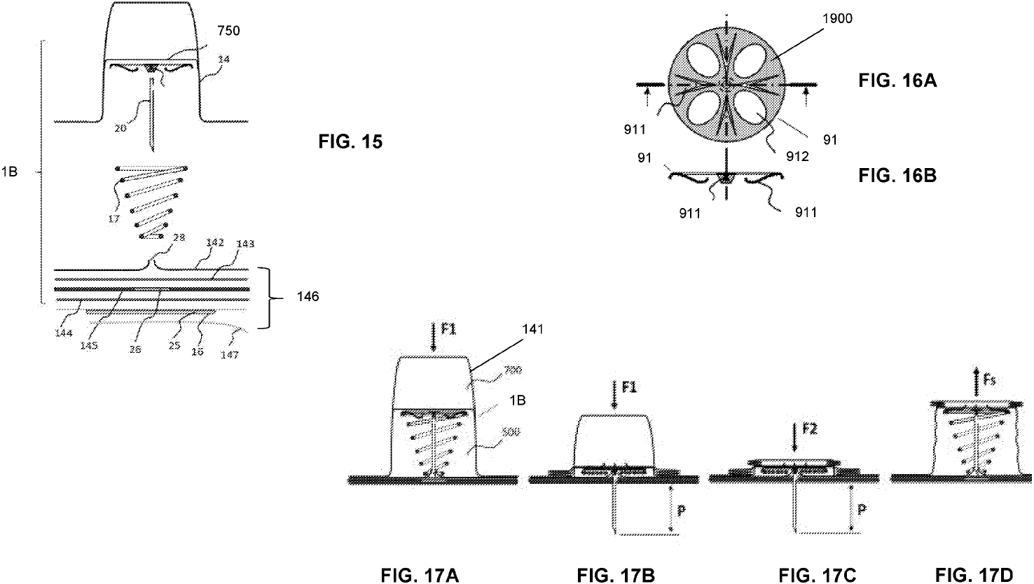

FIGS. 15 to 17D relate to an additional embodiment of an injection capsule according to the invention, globally denoted by 1B.

The capsule 1B comprises a first and a second reservoir, or compartment, 500 and 700 respectively, separated by a hermetic but rupturable, or pierceable, membrane 750.

The second reservoir 700 includes a medical substance.

The first compartment 500 may be used as reservoir of liquid/fluid to be administrated, similarly to second compartment 700, or it may just contain air, which in that case is moved out by a porosity existing in the surrounding collapsible walls.

In addition, the needle holding member, here denoted by 91, incorporates, or is associated with, cutting edges or protrusions 911, preferably with a curved extension according to cords, radiuses or diameters of a retaining plate 1900. The cutting edges 911 are elastically deformable to emerge upwards from plate 1900 so as to face membrane 750.

This embodiment of needle retaining member 91 incorporating cutting edges 911 which are elastically deformable to emerge upwards from plate 1900 can also be applied in the previous embodiments, e.g. in the capsule of FIG. 13. In particular, in this latter case the cutting edges 911 may face the top wall of the main body 14 when the needle 20 reaches its total penetration in the skin. At that moment, the cutting edges 911 create small ruptures in the top wall of main body 14, enough to enable the inlet of air into compartment 400, immediately when the recovery means act to retract the needle and predominantly recover the original shape of collapsible main body 14.

When a force F1 is applied upon body member 141 of capsule 1B and pressure is exercised over the spring element 17, the needle 20 moves downwards, perforating the multi-layered bottom structure 146 and penetrating into the skin, progressively moving up to a defined total penetration P.

When the spring element 17 is completely compressed, the cutting edges 911 are deformed to emerge against membrane 750, perforating and rupturing the latter, so as to open several communication holes or slots between the two compartments 500 and 700. The transfer of a liquid possibly contained in compartment 700 to compartment 500 is facilitated by a plurality of openings 912 in the plate 1900. Keeping applying a force, namely a force F2 greater than previous force F1, the content of reservoir 700 is depleted through the needle 20 and injected. When stopping applying the force, the spring 17 will recover its original shape by a force Fs, pushing the holding plate 1900 up, which brings reservoir 500 to its mostly original shape while completely retracting the needle.

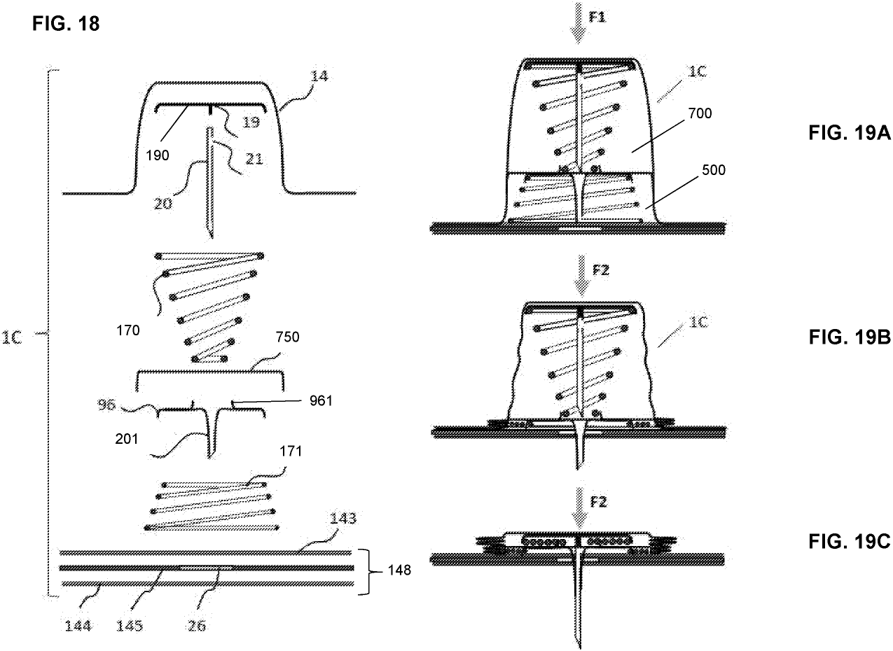

FIGS. 18 to 19C show another variant embodiment of an injection capsule, herein globally denoted by 1C.

In this case, a first and a second needle, respectively 20 and 201, are provided, arranged one coaxially to the other. A first contrast means, in particular a spiral spring denoted by 170, and a second contrast means, in particular a second spiral spring, denoted by 171, are provided, each circumscribed to a respective needle 20, 201. The first spring 170 is arranged similarly to spring 17 of FIG. 13, with its larger section/winding arranged in abutment against the inner surface of the needle retaining plate 190. The second spring 171 has an opposite arrangement, with its larger section/winding in abutment against a base wall 148, having a multilayer but possibly simplified construction with respect to the preceding embodiments.

The injection capsule 1C has, similarly to the preceding embodiment, two compartments 500 and 700 separated by a membrane 750 and each associated with a respective needle 201 and 20. The first compartment 500 may be used as reservoir of liquid/fluid to be administrated, similarly to second compartment 700, or it may just contain air, which in that case is moved out by a porosity existing in the surrounding collapsible walls, and the second compartment 700 a medical substance for final administration.

The second needle 201 has a specially-shaped holding means 96 with spikes 961, possibly made as a single body as the needle itself and configured to perforate membrane 750.

The second needle 201 is slightly larger than the first needle 20 and penetrates the skin first at a first depth. The first needle 20 penetrates deeper for completely injecting the content of the reservoir 700.

This embodiment may be used in cases where standard disinfection means and procedures may not be applicable, as well as when rapid injection is very important while trying to avoid infections as much as possible.

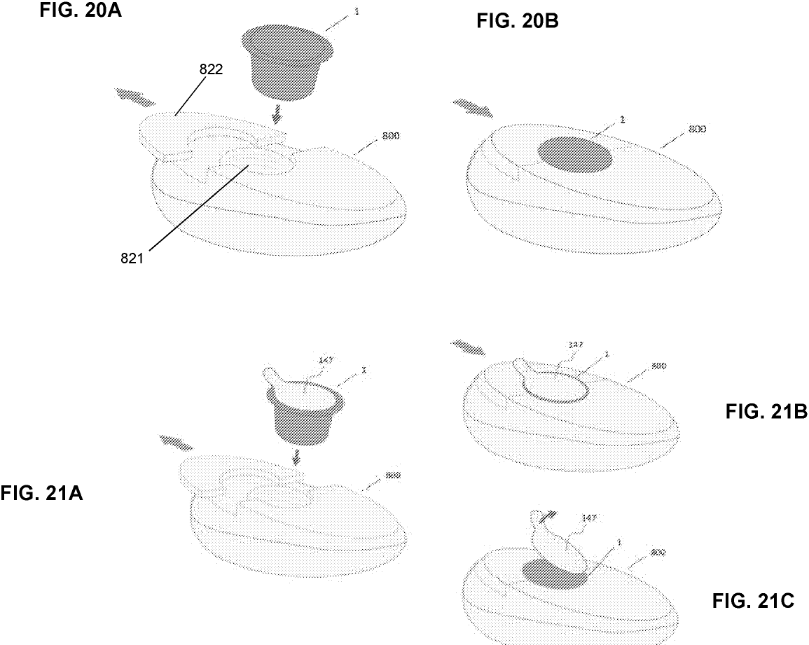

FIGS. 20A and 20B show the injection device 800 according to any of the embodiments presented so far during insertion of a disposable capsule that may be according to any of the embodiments described above. By way of example, capsule 1 according to the variant embodiment of FIG. 5A is represented.

According to the specific embodiment shown, the injection capsule 1 is received within a seat 821 of the injection device 800, which is selectively openable and closable by a closure member 822, in particular a slidable shaped plate which leaves exposed the pierceable structure 146.

Similarly, FIGS. 21A, 21B and 20C show the injection device 800 during insertion of a disposable capsule 1 according to the variant embodiment of FIG. 5 including the peelable external layer 147.

In this case, after insertion the peelable layer 147 is removed.

The embodiments described so far to cover several types of parenteral administration, in variants wherein needles have a straight development and are positioned perpendicular, or substantially perpendicular, to the skin, and follow a straight movement as administration path. Other shapes and orientations of the needle and/or of the administration path covered by the needle itself are possible, both in the previous embodiments and in the ones disclosed below.

For example, there are several advantages in applying a given angle to the needle profile, in order properly administrate the injection in parts of the body where the skin is always very thin and tendons, muscles, blood vessels, or even bones, are in the proximity of the volumetric area that will be penetrated by the needle. The wrists of human body are a good example of such complexity of presence of all those body parts.

In particular, the use of curved needles enables to make a curved penetration in the body and allows to obtain a deviation from less soft, or even rigid parts, of the body that may be encountered on the way of the needle during its penetration, such as tendons and bones. This effect may also be obtained by a natural flexibility in several directions of a curved needle body. The geometry and orientation of a bevel of the needle at the dispensing bore can also be designed to facilitate this effect.

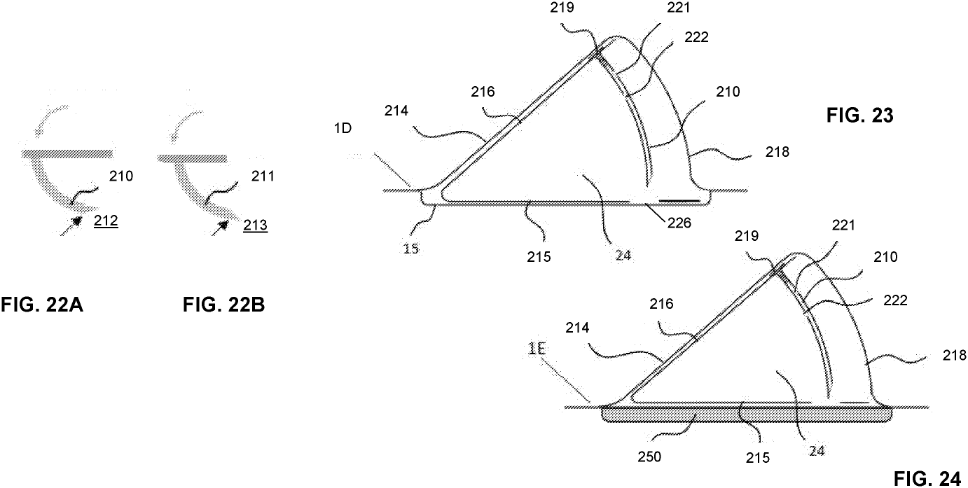

FIGS. 22A and 22B show each, schematically, an embodiment of a curved needle, denoted by 210 and 211, respectively, having a main body with a curved profile. In the variant shown, such main body has an outer profile configured substantially as an arc of circumference, in particular as a quarter of circumference.

The respective dispensing outlet bore 212 and 213 are obtained at oppositely-oriented terminal sections having a bevelled profile.

The orientation of the bevel and of the needle lumen / dispensing outlet bore determines the penetration movement and path.

FIG. 23 shows an embodiment of an injection capsule, globally denoted by 1D, including a curved needle that reproduces the configuration of FIG. 22A and which is therefore globally denoted by 210.

The injection capsule 1D comprises a main body 214, which is globally configured as a flat sector of a cylinder having two lateral inclined walls 215, 216 and a joining curved wall 218. The injection capsule 1D is generally intended to be rested upon the skin with its inclined lateral wall 215. Upon the latter, a through or partial guiding aperture 226 can be obtained, along the needle injection path.

The needle 210 has, upon its curved main body, two inlet bores 221 and 222 obtained at a lateral skirt of said main body. The needle 210 is supported at a needle-holding, or retaining, member 219 fixed with inclined upper wall 216. The capsule main body 214 defines an internal reservoir 24 that hosts the needle 210 and a medical substance.

FIG. 24 shows a variant embodiment of the injection capsule of FIG. 23, herein globally denoted by 1E, it too including a curved needle 210. This embodiment differs from that of FIG. 23 for the presence of a layer 250 applied at the skin and fixed to resting wall 215. Layer 250 may have a similar construction as film structure 146, or of one or more of its components, as disclosed in conjunction with previous embodiments.

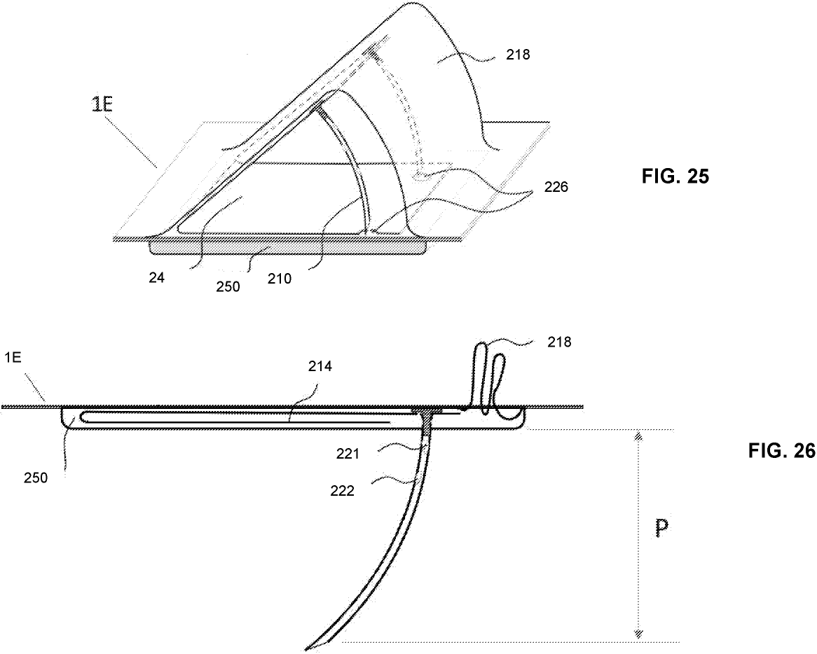

FIGS. 25 and 26 illustrate the operation modes of injection capsule 1E, but are equally applicable for capsule 1D.

As shown in FIG. 25, both embodiments of injections capsule 1D, 1E can incorporate multiple needles, advantageously identical, and preferably arranged so as to perform injection simultaneously, i.e. in parallel.

In FIG. 26 the capsule 1E is shown with main body 214 totally collapsed, which enables to achieve maximum penetration P of needle 210 into the skin.

As shown in the above figures, each of capsules 1D, 1E may be produced by blistering technology.

The same capsules 1D, 1E can be implemented with a double compartment / reservoir. The lower reservoir may contain a disinfectant and/or anaesthetic substance, in liquid or gel form, similarly to what has been disclosed in conjunction with previous embodiments.

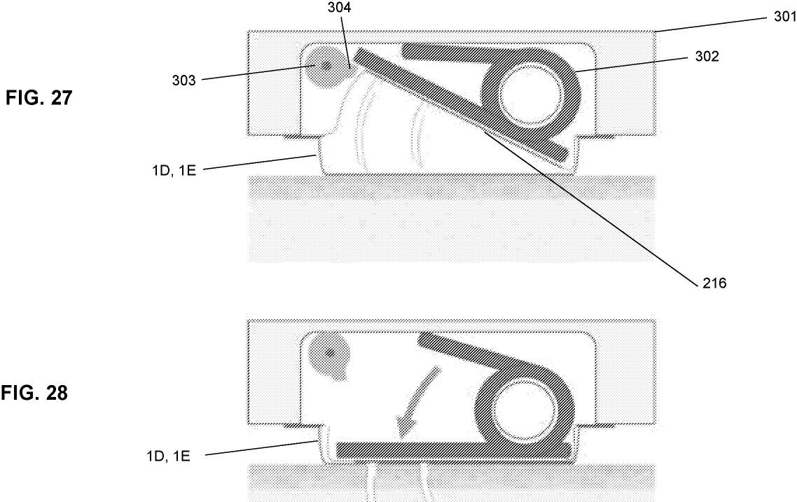

FIGS. 27 and 28 show a further embodiment of an injection device 301 according to the invention, acting upon capsule 1D or 1E.

The injection device 301 incorporates an actuation, or push, member 302 in form of an elastic element arranged in abutment upon upper wall 216. The actuation member 302 is housed within the main body of device 301 in a pre-tensioned, or pre-compressed, condition and released to push and collapse the capsule by an activation, or release, member 303 having an engagement element, e.g. in form of a tooth 304, which retains the actuation member 302 in the pre-tensioned condition by engaging a free end, or arm, thereof. Electronic or manual action upon release member 303 determines disengagement of the actuation member 302, in particular by a rotational movement of release member 303.

FIGS. 30 and 31 show another embodiment of an injection device 401 suitable for use with capsule 1D or 1E, and which has autonomous modular characteristics, as a single module component which can be incorporated in any type of the devices, 800, 500, 50, or in existing wearable devices.

Also in this case, the injection device 401 incorporates a push member 6 in form of a shaped elastic element arranged in abutment upon upper wall 216 with a limb, or arm, 61 thereof.

The push member 6 is housed within a seat or cavity 7 of the device main body in a pre-tensioned, or pre-compressed, condition and released to push and collapse the capsule by an activation, or release, member 8 having an engagement element, e.g. in form of a shaped seat or cavity 84, which retains the release member 6 in the pre-tensioned condition by engaging a free end, or arm, 62 thereof.

Electronic or manual action upon release member 8 determines disengagement of the push member 6, in particular by a rotational movement of release member 8. In the embodiment shown, release member 8 is coupled to a trigger member, in particular a release slider 9, having a protrusion, or tooth, 91 engaging a release seat, or cavity, 81 of release member 8.

When slider 9 translates in a release direction as indicated by an arrow in FIG. 31, the release member 8 rotates and allows disengagement of seat 84 by end 62 of push member 6.

The slider 9 can be associated with an electric / electronic switch, such as a micro-switch controlled by a local control unit 11. The latter may be configured to receive wired signals via a connectivity port, or part, 12, or via wireless means, and act accordingly.

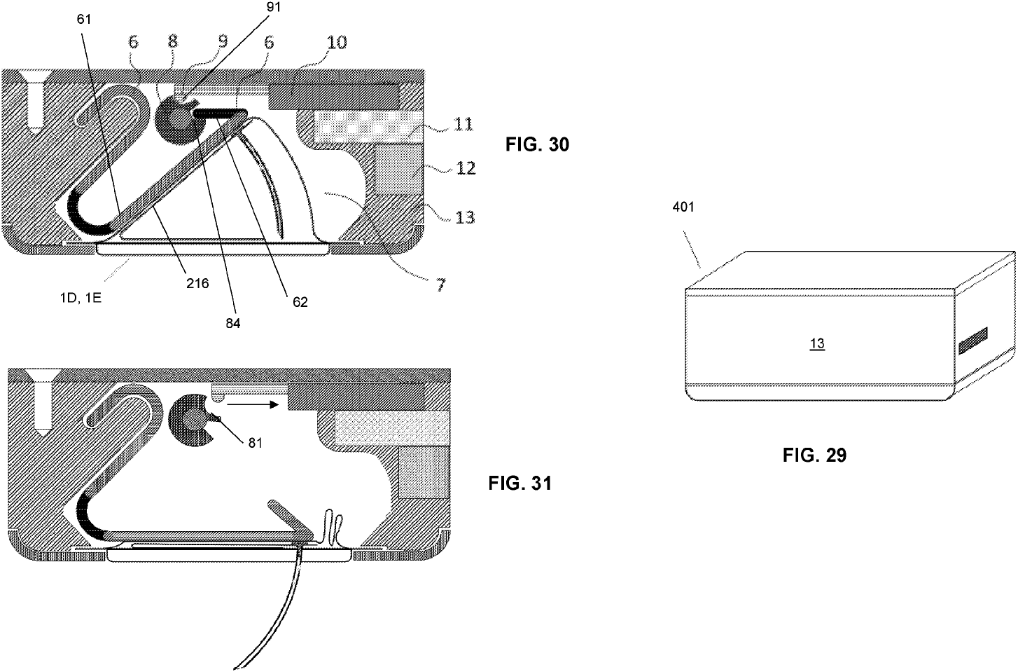

A possible external configuration for main body, or casing, 13 of device 401 is shown in FIG. 29.

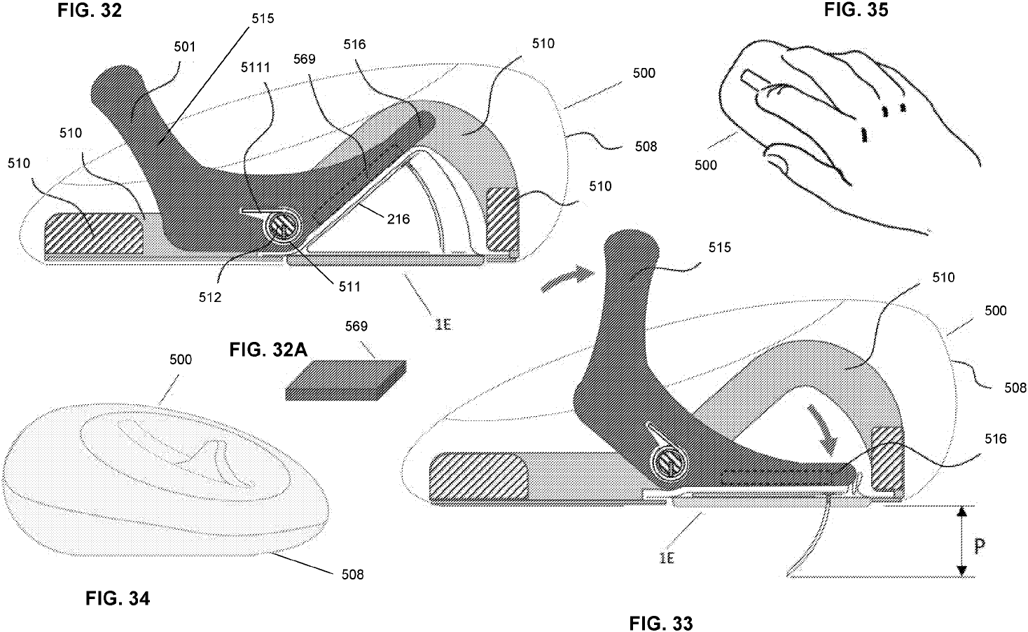

FIGS. 32 to 35 relate to a further embodiment of an injection device, globally denoted by 500, which is suitable to use capsule 1D or 1E. The injection device 500 has a device body 508, or housing, which receives the injection capsule within an internal frame 510.

Also in the present embodiment, the external part of device body 508 is configured substantially like a computer mouse, as better appreciated from FIG. 34. The injection device 500 comprises activation means 501, in form of a rotatable lever or handle, the functioning of which will be explained shortly.

The rotatable lever 501 is maintained in a rest position by a contrast element 511, in particular a helical spring having a terminal arm 5111. The spring 511 is wound, or circumscribed, to a shaft or pivot 512, defining a rotation axis for lever 501. The spring 511 allows the activation means 501 to recover its original position after injection.

The rotatable lever 501 has two main arms, in particular a first arm 515 that can be activated by a human hand or other body district, as shown in FIG. 35, or be driven automatically by other technical means upon a triggering signal provided by a local or remote control unit. A second arm 516 abuts the upper wall 216 of the capsule and pushes it to collapse capsule body during injection.

A recovery means 569, which can be a magnet, or magnetic element, similarly to means 869 in previously presented embodiments - and which is better shown in FIG. 32A – is incorporated into second arm 516 interacting with wall 216. The latter can be made of a material attracted to magnets or electromagnetic generated fields, and/or may incorporate a magnetic element, similarly to the embodiments previously described.

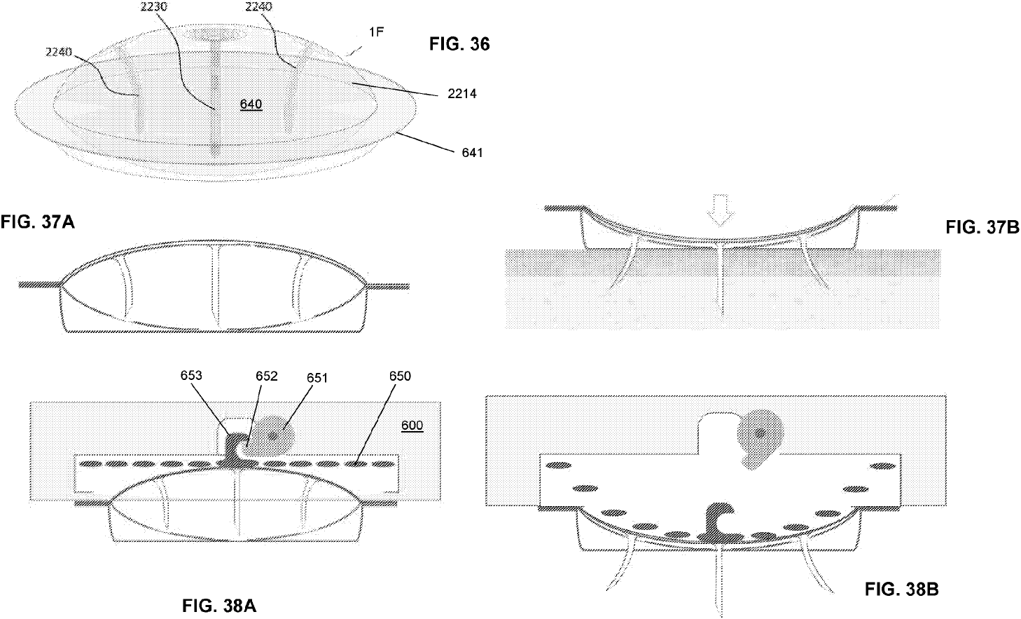

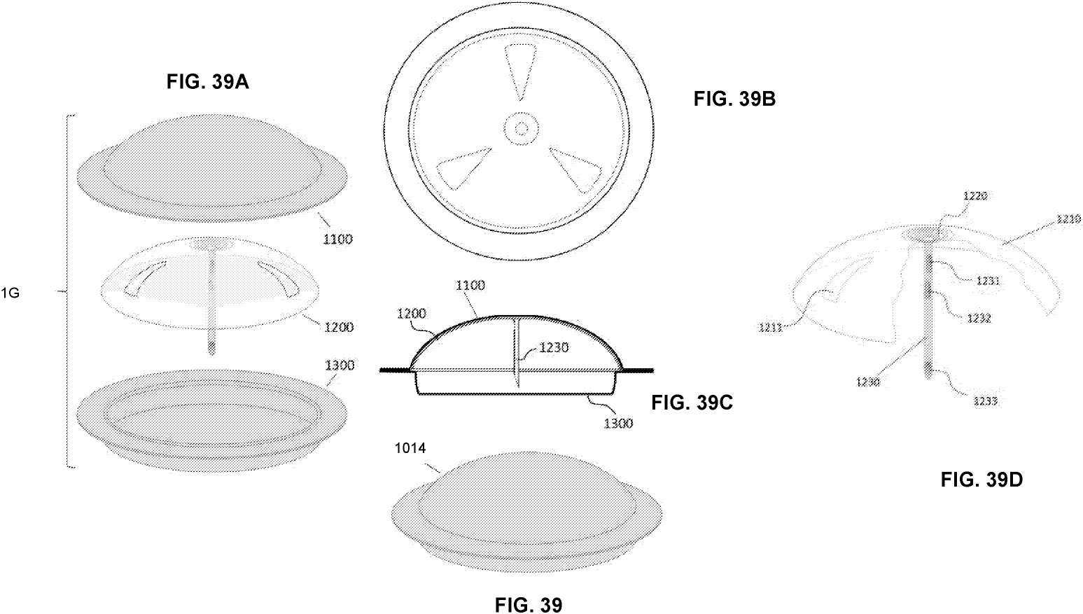

FIGS. 36 to 38B relate to a different embodiment of an injection capsule according to the invention, globally denoted by 1F, which is compatible for use with the injection devices of the previous embodiments.

The injection capsule 1F comprises a main body 2214 configured like a hemispherical shell and defining an internal reservoir 640. A secondary reservoir may be obtained at a peripheral circular flange 641 of the capsule main body.

The main reservoir 640 contains the core liquid for injection and the secondary reservoir 641 may contain a complementary liquid, such as a disinfectant and/or an anaesthetic.

Inside the reservoir 640 three or more needles are housed, in particular a central straight needle 2230 and two lateral curved needles 2240 each having a centrally-converging profile. As in previous embodiments, each needle has one or more inlet bores at a lateral skirt of its elongated main body and a terminal dispensing bore at its longitudinal end.

The cross-section of FIG. 37B shows the capsule 1F after being activated by an axial force which presses the outer shell 2214.

This embodiment of the capsule is particularly suitable for smaller and more compacted applications, as well as when the dose(s) of medication to be administrated is(are) of reduced amount.

The capsule main body, or parts thereof, may be made of standard moulded polymeric compounds, as well as polymeric and metallic foils laminated and blistered.

FIGS. 38A and 38B relate to capsule 1F, shown in conjunction with part of a further embodiment of an injection device, the later globally denoted by 600.

The injection device 600 includes a push means 650 in form of an elastic member, e.g. a spring, for example made of polymeric and/or metallic material. The elastic member 650 can be released by an activation, or release, means 651 having an engagement member 652 couplable with the elastic member 650, e.g. with a hook-shaped appendix 653 of the latter.

The trigger of the release means 651 can be manual or automatic, according to the embodiments already described above.

FIGS. from 39A to 39D relate to a further embodiment of an injection capsule, herein denoted by 1G, that can be used with any of the injection devices described above.

Also in this case, the capsule 1G has a main body 1014 having a mainly hemispherical shape.

The capsule 1G includes an upper component, or shell, 1100.

The capsule 1G further includes a needle-holder, or retaining, member 1200, it too with a hemispherical profile and fixed to the upper shell 1100. Member 1200 is configured with deformation properties such that, when pressed, it reverts its orientation, i.e. its convexity/concavity.

The needle holder 1200 has one or more openings 1210, 1211, which enable the liquid to flow between the two surfaces or parts 1200 and 1100.

The needle is herein straight and denoted by 1230. It has a couple of inlet bores 1231 and 1232, in particular of an elongated shape, at the lateral skirt of its main body, arranged staggered longitudinally and transversely. It also has a dispensing bore 1233 at a terminal section, preferably bevelled.

The needle holder 1200 also a top, central opening 1220 at which the needle 1230 is fastened. Opening 1220 creates a main lumen for the liquid medical substance to flow down through the needle during the operation.

The capsule 1G comprises also a bottom component 1300, which is not collapsible, and its perforated by the needle(s) when the consumable operates. When a force is applied on the top of shell 1100, and consequentially on needle holder member 1200 - such as a force applied by elastic member 650 when released from its compressed state - both parts 1100 and 1200 will progressively deform, collapsing according to their shape, up to completely reverting their geometrical shape, as presented in FIGS. 37B and 38B. During such progressive deformation, the liquid medication is injected, and when reaching such stage the penetration of the central needle 1230 - or 2230 in previous embodiment - reaches its maximum and the injection administration is completed.

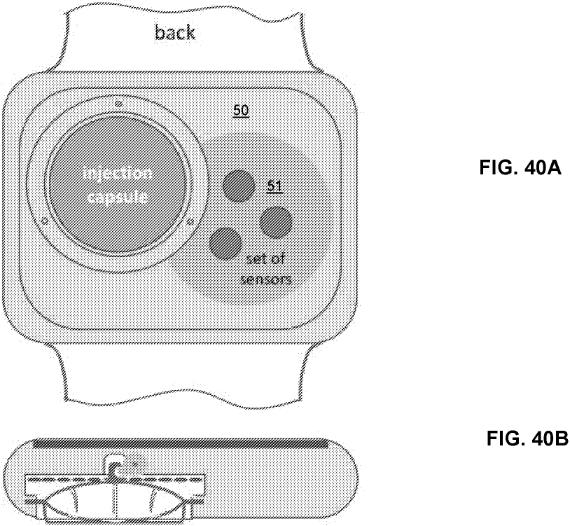

FIGS. 40A and 40B exemplify how an injection device according to any of the previous embodiments and variants can be incorporated into, also as an integral part of, a wearable device 50, such as of a smartwatch type. By way of example, capsule 1F is shown in FIG. 40B.

The wearable device 50 can incorporate sensors 51 to trigger the injection.

It will be better appreciated at this stage that the invention provides, in its various embodiments and variants, one or more of the following advantages.
The injection capsule is an effective substitute of the currently existing sets of syringes and needles for the parenteral administration of medication via injection.
The medical substance(s) included inside the capsule is every time already and there is no need of packaging for the medication, for the syringes and/or for the needles, as well as of all related sterilization procedures and costs during the manufacturing and packaging of those parts/components.
Several types of geometry and size of needles are possible, including curved needles. The needles enable to perform injection covering intradermal injections, subcutaneous injections, as well as reaching intramuscular injections.
Any person can easily use the consumables to administrate medication by injection, without the need of medical/paramedical personnel or of following special procedures.
The capsules may also have integrated means for the disinfection of the area of the skin that will be in contact with the needle, even assuring that at any time during the penetration of the needle the needle goes through a disinfectant.
Different types of liquid medication can be administrated simultaneously in the same single act of injection, while other medication(s) can also be simultaneously applied via the epiderma.
A wide range of uses is possible in medical practices in general, self-practice by individuals, in situations of large number of victims / patients and very few, or not existing at all, access to medical care, means and resources.
A very compact configuration of the capsule and the associated injection device is possible.

The invention is suitable to be applied, e.g., in the medical and paramedical field, in veterinary, military purposes in situations of health monitoring and health management of troops in war fare, as well as management of prisoners, personnel under preventive arrestment by court, and other personnel under high security custody.

The present invention has been described so far with reference to preferred embodiments. It is intended that there may be other embodiments which refer to the same inventive concept as defined by the scope of the following claims.

1. An injection capsule for medical use, comprising:
a closed main body having an internal reservoir receiving at least one medical substance to be administered to a target subject;
a needle, having a tubular body defined by a lateral skirt, said tubular body having a dispensing outlet bore at a first terminal end thereof and at least one inlet bore located at said lateral skirt or at a second terminal end opposed to said first terminal end and configured to allow inlet of the medical substance within the tubular body,
wherein said needle is activatable between a rest configuration, wherein it is housed within said closed main body, and an injection configuration, wherein it is moved outside said closed main body to administer the medical substance by injection to a target subject.

2. The capsule according to claim 1, wherein said main body, or a part thereof, has a collapsible structure configured to assume a minimal encumbrance arrangement in said injection configuration of said needle.

3. The capsule according to claim 1, wherein said needle, in said rest configuration, is housed inside said reservoir, optionally immersed in the medical substance.

4. The capsule according to claim 1,
which comprises recovery means to bring said needle again within said closed main body after injection, which recovery means optionally include one or more of the following: elastic means, in particular spring means; magnetic means, in particular one or more materials that are attracted to magnets or to electromagnetic generated fields, or one or more permanent magnets.

5. The capsule according to claim 4, wherein said recovery means comprises a spirally-shaped spring, or a helically-shaped spring, wound about said needle.

6. The capsule according to claim 1, which comprises a needle holding member arranged at an opposite end with respect to said dispensing outlet bore and optionally having a substantially plate-like or disc-like element from which a needle-engaging element protrudes.

7. The capsule according to claim 4, wherein said recovery means comprise a material that is attracted to a magnet or to an electromagnetic generated field or a magnetic element or part of said needle-holding member.

8. The capsule according to claim 1, wherein said closed main body has a main body member which is substantially cup-like shaped.

9. The capsule according to claim 1, wherein said closed main body has a main body member which is shaped substantially like a cylindrical sector.

10. The capsule according to claim 1, wherein said closed main body has a pierceable base structure, apt to be rested, in use, onto the target body skin.

11. The capsule according to claim 10, wherein said pierceable base structure has a multi-layer construction.

12. The capsule according to claim 10, wherein said pierceable base structure comprises a layer impregnated with a disinfectant and/or with an anaesthetic substance, preferably shielded by a peelable outer film configured to be removed upon use.

13. The capsule according to claim 10, wherein said pierceable base structure comprises an internal reservoir of a disinfectant and/or an anaesthetic substance, optionally configured to be pierced by said needle when activated from said rest configuration to said injection configuration.

14. The capsule according to claim 1, wherein said closed main body includes a plurality of reservoirs, in particular two, each receiving a medical substance to be administered to the target subject or a disinfectant and/or an anaesthetic substance, the injection capsule being preferably configured for a combined or serial administration of said substances.

15. The capsule according to claim 14, wherein the reservoirs of said plurality are separated by a rupturable septum.

16. The capsule according to claim 15, comprising rupturing means arranged within said closed main body and configured to break or pierce said rupturable septum for controlled administration of said substances.

17. The capsule according to claim 1, comprising rupturing means arranged within said closed main body and configured to rupture a wall of said main body after or during injection so as to allow an inlet of air.

18. The capsule according to claim 1, comprising a first and a second needle, arranged one coaxially to the other and slidably coupled to translate one inside the other, the first and the second needle being preferably configured to penetrate a body district of the target subject at different depths.

19. The capsule according to claim 1, comprising a plurality of needles configured to perform injection simultaneously.

20. The capsuleaccording to claim 1, wherein said needle, or at least one needle, has a straight profile, the configuration being optionally such that said needle moves from the rest configuration to the injection configuration according to a translational, or rectilinear, path.

21. The capsule according to claim 1, wherein said needle, or at least one needle, has a curved profile, the configuration being such that the needle moves from the rest configuration to the injection configuration according to a rotational, or arcuate, path.

22. The capsule according to claim 1, which is of a disposable type.

23. An injection device, comprising:
a device body, configured to receive at least one injection capsule according to claim 1; and
activation means, configured to determine activation of said capsule needle from the rest configuration to the injection configuration, which activation means is driven either automatically or by a subject or controlled by a remote interface.

24. The injection device according to claim 23, wherein said activation means include one or more of a slider, a rotatable or push button or an electronic command means or an electronic command means controlled by a remote interface.

25. The injection device according to claim 23, comprising push means configured to move said needle from the rest configuration to the injection configuration, which push means optionally comprises a sliding member, configured to act upon a needle holding member of said injection capsule, or a pre-tensioned or pre-compressed elastic member.

26. The injection device according to claim 23, comprising device recovery means configured to return said needle within the capsule closed main body after injection, which device recovery means preferably comprises one or more of the following: elastic means, in particular spring means; magnetic means, in particular one or more permanent magnets; electromagnetic means.

27. The injection device according to claim 23, comprising a local or remote control unit and/or means for communicating with a local or remote control unit.

28. The injection device according to claim 23, comprising means for detecting a health parameter of the target subject and/or means for communicating with said means for detecting.

29. The injection device according to claim 23, wherein said device body is shaped, substantially like a computer mouse.

30. (canceled)

31. A wearable device, in particular a smart watch, a bracelet, a chest strap or a harm or leg strap, configured to house an injection capsule or an injection device according to claim 1claims and optionally comprising means for detecting a health parameter of the target subject.
